(19) United States
(12) Patent Application Publication
(10) Pub. No.: US 2023/0277582 A1
(43) Pub. Date: Sep. 7, 2023

(54) TREATMENT OF PRIMARY AND/OR SECONDARY LUNG TUMORS USING GASEOUS NITRIC OXIDE INHALATION

(71) Applicant: Beyond Air, Inc., Garden City, NY (US)

(72) Inventors: Hila Confino, Beer Yaakov (IL); Matan Goldshtein, Yavne (IL); Shay Yarkoni, Rehovot (IL); Omer Lerner, Rehovot (IL); Elya Dekel, Rehovot (IL); Shani Puyesky, Rehovot (IL); Steven A. Lisi, Rockville Centre, NY (US); Rinat Kalaora, Rehovot (IL); Amir Avniel, Har Adar (IL)

(21) Appl. No.: 18/115,029

(22) Filed: Feb. 28, 2023

Related U.S. Application Data
(63) Continuation of application No. PCT/IB2021/057857, filed on Aug. 27, 2021.
(60) Provisional application No. 63/071,573, filed on Aug. 28, 2020.

(51) Int. Cl. A61K 33/00; A61P 35/00; A61K 9/00; A61K 45/06

(52) U.S. Cl. CPC A61K 33/00; A61P 35/00; A61K 9/007; A61K 45/06

(57) ABSTRACT

Described herein are methods of inhibiting growth of cells or tissue of a primary and/or secondary tumor in a respiratory tract of a subject in need thereof and/or sensitizing cells or tissue of a primary and/or secondary tumor in a respiratory tract of the subject to an anti-cancer therapy. The methods are effected by administering gaseous nitric oxide (gNO) via inhalation at a concentration in a range of from about 1 ppm to about 1,000 ppm. The gNO may be co-administered with an anti-cancer therapy and/or an agent suitable for treating methemoglobinemia.

### RELATED APPLICATION

This application is a continuation of International Application No. PCT/IB2021/057857, which designated the United States and was filed on Aug. 27, 2021, published in English, which claims the benefit of U.S. Provisional Application No. 63/071,573, filed on Aug. 28, 2020. The entire teachings of the above applications are incorporated herein by reference.

### FIELD AND BACKGROUND OF THE INVENTION

The present invention generally relates to therapy, and more particularly, but not exclusively, to novel methodologies which employ gaseous nitric oxide (gNO) for inhibiting growth of cells or tissue of a primary and/or secondary tumor in the respiratory tract.

Lung and bronchus cancers are a leading cause of cancer deaths. In addition, metastasis is responsible for a large portion of cancer deaths, and many cancers metastasize to the lung. Common cancers that metastasize to lungs include breast cancer, lung cancer, colorectal cancer, uterine leiomyosarcoma, and head/neck squamous cell carcinomas, renal cancer, lung cancer, osteosarcoma, testicular cancer, and lymphomas.

Currently, the typical systemic anti-metastatic treatment is chemotherapy, which mostly reacts against all replicating cells and therefore considered as a non-specific therapy. However, resting cancer cells will not be destroyed by this treatment. An alternative approach is to attempt to induce the host immune system to react against tumor-associated antigens, for example, by ablation of the tumor that results in the release of tumor antigens which trigger the immune system [Keisari et al., Cancer Immunol Immunother 2014, 63:1-9].

Programmed death-ligand 1 (PDL1) on cancer cells engages the immune checkpoint molecule programmed cell death protein 1 (PD1) on immune cells, contributing to escape from the immune system. Blocking PD1/PDL1 could restore T cells from exhausted status and eradicate cancer cells, but only some PDL1-positive patients benefit from α-PD1/PDL1 therapies [Yi et al., J Hematol Oncol 2021, 14:10]. Moreover, the pressure of PD1/PDL1 blockade may transform immune checkpoint inhibitor-sensitive tumors into resistant tumors [Pathak et al., Cancers (Basel) 2020, 12:3851].

Nitric oxide (NO) is a short-lived, endogenously produced gas that acts as a signaling molecule in the body. Induction of vasodilation by NO released by endothelial cells is a well-known function of NO. In addition, interaction of NO with O₂ or O₂⁻ results in reactive oxygen species that can act as chemical stressors of cells. NO secreted as part of an immune response can be toxic to bacteria and other cells.

Huerta [Futur Sci OA 2015, FSO44] has reported that at high doses, NO has an antineoplastic effect, and can be used in cancer therapy either as a single agent or in combination with other neoplastic compounds.

Bonavida & Garban [Redox Biol 2015, 6:486-494] has reported that NO mediates sensitization of resistant tumor cells to apoptosis by chemotherapeutic drugs and cytotoxic immune responses.

NO donors have been reported to induce tumor cell death via apoptosis [Shang et al., J Oral Maxillofac Surg 2002, 60:905-910; Kiziltepe et al., Blood 2007, 110:709-718] and necrosis [Weyerbock et al., J Neurosurg 2009, 110:128-136]; and to sensitize cancer cells to chemotherapy [Alimoradi et al., Int J Nanomedicine 2018, 13:7771-7787] and radiotherapy [Scicinski et al., Redox Biol 2015, 6:1-8]. Overexpression of inducible nitric oxide synthase has been reported to inhibit cancer cell migration in vitro and metastases in vivo [Harada et al., In Vivo 2004, 18:449-455]. In addition, sildenafil, a phosphodiesterase 5 (PDE5) inhibitor that enhances NO signaling, has been reported to potentiate the antitumor activity of cisplatin by induction of apoptosis and inhibition of proliferation and angiogenesis [El-Naa et al., Drug Des Devel Ther 2016, 10:3661-3672].

Although NO may exhibit a beneficial effect in cancer by being cytotoxic and/or an antioxidant, NO may also be a negative prognostic indicator in cancer due to its ability to enhance angiogenesis, stimulate migration and invasion, and induce DNA damage; wherein the difference may depend on NO concentration and the type and distribution of cellular targets [Thomas, Redox Biol 2015, 5:225-233].

Inhalation of gaseous nitric oxide (gNO) has been studied in connection with respiratory diseases. Inhaled NO is a common treatment of persistent pulmonary hypertension of the newborn.

U.S. Pat. Nos. 5,485,827 and 5,873,359 describe devices and methods for treating or preventing bronchoconstriction or reversible pulmonary vasoconstriction in a mammal, effected by causing the mammal to inhale a therapeutically-effective concentration of NO in a gaseous form or a therapeutically-effective amount of an NO-releasing compound, and an inhaler device containing NO gas and/or an NO-releasing compound.

U.S. Patent Application Publication No. 2010/0051025 describes systems, compositions and methods for preventing or reducing vasoconstriction in a mammal, involving administering to a mammal a composition containing an artificial oxygen carrier in combination with one or more of an NO-releasing compound, a therapeutic gas containing NO, a phosphodiesterase inhibitor, and/or a soluble guanylate cyclase sensitizer.

Gaseous NO has also been investigated for use in antimicrobial treatments. At 200 ppm, gNO reduced S. aureus burden in a rabbit wound model without being cytotoxic to human fibroblast, keratinocyte, endothelial, monocyte and macrophage cells in culture [Schairer et al., Virulence 2012, 3:271-279].

International Patent Application Publication WO 2014/136111 describes intermittent inhalation of gaseous NO at a concentration of at least 160 ppm as being suitable for treating a disease or disorder manifested in the respiratory tract, such as an infection in an immune-compromised subject (including cancer patients undergoing chemotherapy).

International Patent Application Publication WO 2021/105901 describes a method of inhibiting growth of cells or tissue of a primary and/or secondary tumor, and/or of stimulating an immunological response to the tumor, by employing local administration of a gas, such as gaseous NO, typically at a high dose.

International Patent Application Publication WO 2015/037002 describes a system for inhalation of a mixture of NO and a carrier gas mixture which contains O₂.

NO inhalation may lead to methemoglobin production due to oxidation of hemoglobin by NO. High levels of methemoglobin are often treated using methylene blue; unless the patient is a small infant or has glucose-6-phosphate dehydrogenase deficiency, in which case ascorbic acid may be used [Moughnyeh et al., J Pediatr Surg Case Rep 2020, 57:101457].

Additional background art includes Lin et al. [Adv Sci (Weinheim) 2019, 6:1802062]; Niedbala et al. [Ann Rheum Dis 2006, 65:iii37-iii40]; Seabra & Duran [Eur J Pharmacol 2018, 826:158-168]; Vannini et al. [Redox Biol 2015, 6:334-343]; and International Patent Application Publications WO 2009/036571, WO 2011/141863, WO 2012/153331, WO 2013/132497, WO 2013/132498, WO 2013/132499, WO 2013/132500, WO 2013/132503 and WO 2021/105900.

### SUMMARY OF THE INVENTION

According to an aspect of some embodiments of the invention, there is provided a method of inhibiting growth of cells or tissue of a primary and/or secondary tumor in a respiratory tract of a subject in need thereof, the method comprising administering to the subject gaseous nitric oxide (gNO) via inhalation for at least 1 second per day, wherein a concentration of gNO is in a range of from about 1 ppm to about 1,000 ppm.

According to an aspect of some embodiments of the invention, there is provided a method of inhibiting growth of cells or tissue of a primary and/or secondary tumor in a respiratory tract of a subject in need thereof, the method comprising co-administering to the subject an anti-cancer therapy and gaseous nitric oxide (gNO), the gNO being administered via inhalation for at least 1 second per day, wherein a concentration of gNO is in a range of from about 1 ppm to about 1,000 ppm.

According to an aspect of some embodiments of the invention, there is provided a method of sensitizing cells or tissue of a primary and/or secondary tumor in a respiratory tract of a subject in need thereof to an anti-cancer therapy, the method comprising co-administering to the subject the anti-cancer therapy and gaseous nitric oxide (gNO), the gNO being administered via inhalation for at least 1 second per day, wherein a concentration of gNO is in a range of from about 1 ppm to about 1,000 ppm.

According to an aspect of some embodiments of the invention, there is provided a method of inhibiting growth of cells or tissue of a primary and/or secondary tumor in a respiratory tract of a subject in need thereof, the method comprising co-administering to the subject gaseous nitric oxide (gNO), the gNO being administered via inhalation for at least 1 second per day, wherein a concentration of gNO is in a range of from about 1 ppm to about 1,000 ppm, and an agent suitable for treating methemoglobinemia.

According to some of any of the embodiments described herein, the concentration of gNO is in a range of from about 10 ppm to about 600 ppm.

According to some of any of the embodiments described herein, the concentration of gNO is in a range of from about 10 ppm to about 250 ppm.

According to some of any of the embodiments described herein, the concentration of gNO is in a range of from about 250 ppm to about 600 ppm.

According to some of any of the embodiments described herein, the method comprises administering gNO for at least 10 minutes per day.

According to some of any of the embodiments described herein, the method comprises administering gNO for no more than about 600 minutes per day.

According to some of any of the embodiments described herein, the method comprises administering gNO from about 30 to about 120 minutes per day.

According to some of any of the embodiments described herein, the gNO is mixed with a carrier gas, and a volumetric flow of the gNO mixed with the carrier gas is from about 1 liter per minute to about 100 liters per minute.

According to some of any of the embodiments described herein, the gNO is mixed with a carrier gas, and a volumetric flow of the gNO mixed with the carrier gas is from about 5 liter per minute to about 20 liters per minute.

According to some of any of the embodiments described herein, the method comprises administering gNO for a time period in a range of from 1 to 100 days.

According to some of any of the embodiments described herein, the method comprises administering gNO for a time period in a range of from 7 to 21 days.

According to some of any of the embodiments described herein, the method comprises administering gNO more than once per day.

According to some of any of the embodiments described herein, the method comprises administering gNO from 1 to 6 times per day.

According to some of any of the embodiments described herein, the method comprises administering gNO from 1 to 4 times per day.

According to some of any of the embodiments described herein relating to administration of gNO from 1 to 4 times per day, the gNO is administered from about 1 minute to about 180 minutes independently in each of the 1 to 4 times.

According to some of any of the embodiments described herein, the method comprises administering gNO 4 times per day.

According to some of any of the embodiments described herein relating to administration of gNO 4 times per day, the gNO is administered from about 1 minute to about 60 minutes independently in each of the 4 times.

According to some of any of the embodiments described herein, the method comprises administering gNO 2 times per day.

According to some of any of the embodiments described herein relating to administration of gNO 2 times per day, the gNO is administered from about 1 minute to about 90 minutes independently in each of the 2 times.

According to some of any of the embodiments described herein, a product of the concentration of gNO and a daily time of inhalation of gNO is in a range of from about 0.0027 ppm·hour to about 6000 ppm·hour.

According to some of any of the embodiments described herein, the primary and/or secondary tumor in a respiratory tract is a lung cancer tumor and/or a lung metastasis.

According to some of any of the embodiments described herein, the method further comprises administering an agent suitable for treating methemoglobinemia.

According to some of any of the respective embodiments described herein, agent suitable for treating methemoglobinemia comprises methylene blue.

According to some of any of the embodiments described herein, the method further comprises co-administering to the subject an anti-cancer therapy.

According to some of any of the embodiments described herein relating to an anti-cancer therapy, the anti-cancer therapy is selected from the group consisting of a chemotherapeutic agent, an immune-oncological agent, and a radiation treatment.

According to some of any of the embodiments described herein relating to a chemotherapeutic agent, the chemotherapeutic agent is selected from the group consisting of 5-fluorouracil and an EGFR tyrosine kinase inhibitor.

According to some of any of the embodiments described herein relating to an immune-oncological agent, the immune-oncological agent is a PD1 inhibitor and/or a PDL1 inhibitor.

According to some of any of the embodiments described herein relating to an anti-cancer therapy, the anti-cancer therapy is administered at a sub-therapeutic dosage.

According to some of any of the embodiments described herein relating to an anti-cancer therapy, administering the gNO is performed prior to, subsequent to and/or concomitant with administering the anti-cancer therapy.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents a bar graph showing the viability of CT26 colon cancer cells upon exposure to 400 ppm NO or to air, for 1 hour.

FIGS. 2A-2C present bar graphs showing the viability of CT26 colon cancer cells 24 hours after one (FIG. 2A), four (FIG. 2B) or seven (FIG. 2C) cycles of exposure to NO for 15 minutes (results are normalized to non-treated cells; n=3; *p<0.01, ***p<0.001, ****p<0.0001, determined by Student's t-test).

FIG. 3 presents a bar graph showing the viability of LLC1 lung cancer cells upon exposure for 15 or 30 minutes to 200 ppm NO or to air.

FIG. 4 presents a bar graph showing the viability of CT26 colon cancer cells upon exposure for 1 μM 5-fluorouracil (5FU) and/or 30 minutes to 200 ppm NO.

FIG. 5 presents a bar graph showing the viability of CT26 colon cancer cells after four cycles of exposure to 200 ppm NO for 10 minutes, followed immediately by addition of 0.5, 1, 2.5 or 50 μM 5-fluorouracil (5FU); for comparison, cells were exposed to air or not placed in a gas exposure chamber (control), and/or not treated (NT) with 5FU (results are normalized to non-treated cells; n=3; *p<0.05, determined by Student's t-test).

FIG. 6 presents a graph showing survival of mice as a function of time after induction of lung metastasis by injection of CT26 cancer cells, upon administration via inhalation of 200 ppm NO or air (on days 3-18), with or without administration of 20 mg/kg 5FU (on days 4, 11 and 16) (P value for Kaplan Meier log rank test—0.013).

FIG. 7 illustrates an exemplary system for administering gNO via inhalation, according to some embodiments of the present invention.

### DESCRIPTION OF SPECIFIC EMBODIMENTS OF THE INVENTION

The present invention generally relates to therapy, and more particularly, but not exclusively, to novel methodologies which employ gaseous nitric oxide (gNO) for inhibiting growth of cells or tissue of a primary and/or secondary tumor in the respiratory tract.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have uncovered that administration via inhalation of gaseous nitric oxide can be used to treat a wide variety of respiratory tract tumors, including primary lung tumors and lung metastases; and that the nature of such a treatment, involving diffusion of gas through the respiratory tract, may combine advantageous features of local administration (e.g., minimal side effects associated with systemic administration of an active agent) and advantageous features of systemic administration, for example, the ability to treat inoperable tumors and tumors, multiple tumors (e.g., metastases), as well as tumors of any size and shape (including difficult to operate flat and amorphous tumors).

According to an aspect of some embodiments of the invention, there is provided a method of inhibiting growth of cells or tissue of a primary and/or secondary tumor in a subject in need thereof (e.g., a mammalian subject, preferably a human subject), the method comprising administering to the subject gaseous nitric oxide (gNO) via inhalation.

According to an aspect of some embodiments of the invention, there is provided gaseous nitric oxide (gNO) for use in the treatment of a primary and/or secondary tumor (e.g., the treatment comprising inhibiting growth of cells or tissue of the tumor in a subject in need thereof).

According to an aspect of some embodiments of the invention, there is provided a use of gaseous nitric oxide (gNO) in the manufacture of a medicament for treatment of a primary and/or secondary tumor (e.g., the treatment comprising inhibiting growth of cells or tissue of the tumor in a subject in need thereof).

According to some of any of the embodiments described herein, the treatment comprises administering the gNO via inhalation for at least 1 second per day, as described in further detail hereinunder.

According to some of any of the embodiments described herein, administering the gNO via inhalation is for at least 1 second per day, as described in further detail hereinunder.

In some of any of the respective embodiments described herein, a concentration of gNO is no more than about 1,000 ppm (e.g., from about 1 ppm to about 1,000 ppm); for example, where the remainder may be any suitable carrier gas (e.g., air, an N₂/O₂ mixture, and/or any other gas mixture which is suitable for inhalation and does not react with NO), which optionally comprises about 21% O₂. In some such embodiments, the concentration of gNO is no more than 600 ppm (e.g., from about to about 600 ppm, or from about 150 to about 600 ppm, or from about 250 to about 600 ppm). In some such embodiments, the concentration of gNO is no more than 250 ppm (e.g., from about 10 to about 250 ppm, or from about 150 to about 250 ppm). Exemplary concentration of gNO include about 200 ppm and about 400 ppm.

Herein, the term “ppm” refers to parts per million by volumetric fraction.

The concentration of gNO may optionally fluctuate over time. Thus, gNO concentrations described herein may be regarded as an average concentration over time.

In some of any of the respective embodiments, a volumetric flow of the gas comprising gNO (e.g., gNO mixed with a carrier gas according to any of the respective embodiments described herein) is at least about 1 liter per minute (e.g., from about 1 to 200 liters per minute, or from about 1 to 100 liters per minute, or from about 1 to about 20 liters per minute), optionally at least about 5 liters per minute (e.g., from about 5 to 200 liters per minute, or from about 5 to 100 liters per minute, or from about 5 to about 20 liters per minute), or at least about 60 liters per minute (e.g., from about 60 to 200 liters per minute, or from about 60 to 100 liters per minute). In some embodiments, the flow is about 15 to about 20 liters per minute for an adult human, and about 5 liters per minutes for a child.

The method and/or treatment may optionally comprise administration on a single day and/or on multiple days, for example, for 100 days or more. When administration is effected on multiple days, the days may be consecutive days or non-consecutive days.

In some of any of the respective embodiments, administration is effected for a time period of from 1 to 100 days (e.g., consecutive days). In some embodiments, administration is effected for a time period of at least 7 days (e.g., consecutive days), optionally from 7 to 21 days (e.g., 7 or 14 or 21 consecutive days). Administration on each day may be effected once or more than once, e.g., according to any of the respective embodiments described herein.

In some of any of the respective embodiments, the gNO is administered for at least about 1 minute per day (e.g., up to 24 hours per day), and optionally for at least about 5 minutes per day, or for at least about 10 minutes per day, or for at least about 15 minutes per day, or for at least about 20 minutes per day, for at least about 30 minutes per day, or for at least about 60 minutes per day.

The gNO may optionally be administered continuously, essentially 24 hours per day (optionally with short breaks, e.g., for eating, going to the bathroom, and the like), such that the duration of exposure to gNO is the number of days during which the treatment is effected. Alternatively, gNO is administered for no more than about 600 minutes per day (e.g., from about 1 to about 600 minutes per day, or from about 10 to about 600 minutes per day, or from about 30 to about 600 minutes per day, or from about 60 to about 600 minutes per day), and optionally for no more than about 240 minutes per day (e.g., from about 1 to about 240 minutes per day, or from about 10 to about 240 minutes per day, or from about 30 to about 240 minutes per day, or from about 60 to about 240 minutes per day), or no more than about 120 minutes per day (e.g., from about 1 to about 120 minutes per day, or from about 10 to about 120 minutes per day, or from about 30 to about 120 minutes per day, or from about 60 to about 120 minutes per day).

Administration of gNO may optionally be effected continuously, i.e., once per day, e.g., such that a time of administration per day according to any of the respective embodiments described herein represents a single continuous administration. Continuous administration may optionally be effected, for example, for up to about 12 hours (e.g., from about 6 to about 12 hours), optionally using a gNO concentration in a range of from about 150 to about 600 ppm (e.g., about 200 ppm or about 400 ppm). Alternatively or additionally, administration of gNO may optionally be effected intermittently, i.e., more than once per day; e.g., such that a time of administration per day according to any of the respective embodiments described herein represents a sum of two or more separate administration periods, which may be of the same length or of different lengths. For example, administration of gNO for 60 minutes per day may optionally be effected as two separate 30 minute administrations, three separate 20 minute administrations, four separate 15 minute administrations, and so forth. For intermittent administration, the more than one administration periods in the same day may optionally be separated from one another by a pause of at least 5 minutes, or at least 20 minutes, or at least 1 hour, or at least 2 hours.

In some of any of the respective embodiments described herein, gNO is administered from 1 to 6 times per day (e.g., from 2 to 6 times per day), and optionally from 1 to 4 times per day (e.g., from 2 to 4 times per day). In some such embodiments, each of the aforementioned 1 to 6 (or 1 to 4) administrations is independently from about 1 minute to about 180 minutes (e.g., from about 1 to about 90 minutes). In some such embodiments, each of the administrations is independently from about 2 to about 180 minutes (e.g., from about 2 to about 90 minutes), or from about 5 to about 180 minutes (e.g., from about 5 to about 90 minutes), or from about 10 to about 180 minutes (e.g., from about 10 to about 90 minutes). In some of any of the aforementioned embodiments, administration is effected for a time period of from 7 to 21 days (e.g., 7 or 14 or 21 days), according to any of the respective embodiments described herein.

In some of any of the respective embodiments described herein, gNO is administered four times per day, optionally wherein each of the four administrations per day is independently from about 1 minute to about 60 minutes (e.g., from about 1 to about 30 minutes). In some such embodiments, each of the four administrations per day is independently from about 2 to about 60 minutes (e.g., from about 2 to about 30 minutes), or from about 3 to about 60 minutes (e.g., from about 3 to about 30 minutes), or from about 5 to about 60 minutes (e.g., from about 5 to about 30 minutes).

In some of any of the respective embodiments described herein, gNO is administered twice per day, optionally wherein each of the two administrations per day is independently from about 1 minute to about 90 minutes (e.g., from about 1 to about 60 minutes). In some such embodiments, each of the two administrations per day is independently from about 2 to about 90 minutes (e.g., from about 2 to about 60 minutes), or from about 5 to about 90 minutes (e.g., from about 5 to about 60 minutes), or from about 10 to about 90 minutes (e.g., from about 10 to about 60 minutes).

The dosage of gNO may optionally be characterized as a product of the gNO concentration and the time of administration (e.g., time of administration per day), expressed in units of concentration multiplied by time (e.g., ppm·hour).

Thus, in some of any of the respective embodiments described herein, the daily dosage of gNO is at least about 0.0027 ppm·hour, for example, from about 0.0027 to about 6000 ppm·hour, or from about 0.0027 to about 1200 ppm·hour, or from about 0.0027 to about 500 ppm·hour. In some such embodiments, the daily dosage of gNO is at least about 0.17 ppm·hour, for example, from about 0.17 to about 6000 ppm·hour, or from about 0.17 to about 1200 ppm·hour, or from about 0.17 to about 500 ppm·hour. In some embodiments, the daily dosage of gNO is at least about 1 ppm·hour, for example, from about 1 to about 6000 ppm·hour, or from about 1 to about 1200 ppm·hour, or from about 1 to about 500 ppm·hour. In some embodiments, the daily dosage of gNO is at least about 5 ppm·hour, for example, from about 5 to about 6000 ppm·hour, or from about 5 to about 1200 ppm·hour, or from about 5 to about 500 ppm·hour. In some embodiments, the daily dosage of gNO is at least about 25 ppm·hour, for example, from about 25 to about 6000 ppm·hour, or from about 25 to about 1200 ppm·hour, or from about 25 to about 500 ppm·hour. In some embodiments, the daily dosage of gNO is at least about 125 ppm·hour, for example, from about 125 to about 6000 ppm·hour, or from about 125 to about 1200 ppm·hour, or from about 125 to about 500 ppm·hour.

For example, for a daily dosage of 600 ppm·hour may optionally be effected by administering gNO twice per day, wherein each of the two administrations is at a dosage of 300 ppm·hour (alternatively, the daily dosage may be divided into two unequal dosages). Each administration of 300 ppm·hour may comprise, for example, administering gNO at a concentration of 600 ppm for 30 minutes, or at a concentration of 300 ppm for 1 hour, or at a concentration of 150 ppm for 2 hours.

Similarly, a daily dosage of 600 ppm·hour may optionally be effected by administering gNO four times per day, wherein each of the four administrations is at a dosage of 150 ppm·hour (alternatively or additionally, at least some of the four administrations may comprise different dosages). Each administration of 150 ppm·hour may comprise, for example, administering gNO at a concentration of 600 ppm for 15 minutes, or at a concentration of 300 ppm for 30 minutes, or at a concentration of 150 ppm for 1 hour.

It will be appreciated that gNO inhalation may result in an increase in methemoglobin levels (associated with oxidation of hemoglobin iron by NO), which may represent a clinical limitation on the amount of gNO which may be inhaled.

Thus, for some of any of the respective embodiments described herein (especially embodiments involving a relatively high dosage of gNO), the method preferably further comprises monitoring a methemoglobin level in the blood of the subject after administration of gNO is initiated, optionally during administration of gNO. If methemoglobin levels rise above a predetermined threshold (e.g., 3, 4 or 5%), gNO administration may optionally be ceased, and optionally restored only after decrease of methemoglobin levels to well below the threshold. For example, intermittent administration of gNO (e.g., according to any of the respective embodiments described herein) may optionally be timed (e.g., with respect to the duration of each administration and/or the pauses between administrations) to avoid excess methemoglobin levels.

The present inventors have conceived that combining gNO inhalation with administration of an agent suitable for treating methemoglobinemia (according to any of the respective embodiments described herein) may allow administering higher dosages of gNO (e.g., higher concentrations and/or longer durations of administration), for example, without methemoglobin levels rising above a predetermined threshold (e.g., 3, 4 or 5%).

In some of any of the respective embodiments described herein, the method and/or treatment further comprises administering an agent suitable for treating methemoglobinemia. Co-administration of the agent suitable for treating methemoglobinemia may optionally be performed prior to, subsequent to, and/or concomitant with the gNO administration; for example, shortly prior to (e.g., no more than 1 hour or no more than 10 minutes prior to) and/or concomitant with the gNO administration.

Any agent capable of enhancing reduction of methemoglobin in vivo (e.g., by NADPH methemoglobin reductase) at a concentration which is not excessively toxic may be used. Examples of agents suitable for treating methemoglobinemia include, without limitation, methylene blue and ascorbic acid. Methylene blue is an exemplary agent for treating methemoglobinemia, and may optionally be administered intravenously (e.g., as an aqueous solution of from 2 to 50 mg/ml, of from 5 to 20 mg/kg, or about 10 mg/kg), for example, at a methylene blue dosage in a range of from 0.2 to 10 mg/kg, or from 0.5 to 4 mg/kg, or from 1 to 2 mg/kg.

Without being bound by any particular theory, it is believed that agents which accept electrons from NADPH methemoglobin reductase are particularly effective at enhancing reduction of methemoglobin by enhancing the activity of NADPH methemoglobin reductase in red blood cells (which tend to be deficient of such an electron acceptor).

In some of any of the respective embodiments described herein, administration of agent suitable for treating methemoglobinemia facilitates use of relatively high dosages of gNO; for example, gNO concentrations of about 150 ppm or more (according to any of the respective embodiments described herein), gNO administration times of about 10 minutes per day or more (according to any of the respective embodiments described herein), and/or daily gNO dosages of about 5 ppm·hour or more (according to any of the respective embodiments described herein).

In some of any of the respective embodiments described herein, the method comprises monitoring a methemoglobin level of the subject after administration of gNO is initiated, and optionally administering an agent suitable for treating methemoglobinemia (according to any of the respective embodiments described herein) if methemoglobin levels rise above a predetermined threshold (e.g., 3, 4 or 5% or total hemoglobin).

In some of any of the respective embodiments described herein, the method comprises monitoring a methemoglobin level of the subject after administration of gNO is initiated, and optionally administering an agent suitable for treating methemoglobinemia (according to any of the respective embodiments described herein) if methemoglobin levels rise above a predetermined threshold as described herein, whereby administration of gNO is effected, for example, at a concentration as described herein in any of the respective embodiments and continuously for a time period of 2 hours, 3, hours, 4, hours, 5 hours, 6 hours, 7 hours, 8 hours, and even more (e.g., while periodically monitoring the methemoglobin level and administering an agent suitable for treating methemoglobinemia as needed, as described herein).

As used herein throughout, the term “tumor” describes a plurality of cells or a tissue composed of the plurality of cells that are characterized by abnormal cell growth and which serve no physiological function. The term “tumor” is also referred to herein and in the art as “neoplastic tissue, and encompasses benign, pro-malignant and malignant tumors.

By “abnormal cell growth” it is meant uncontrolled, progressive proliferation of the cells, which is no longer under normal bodily control. The growth of a tumor tissue typically exceeds, and is uncoordinated with, that of the normal cells or tissues around it.

“Abnormal cell growth” also describes cell growth that is independent of normal regulatory mechanisms (e.g., loss of contact inhibition), including, for example, abnormal growth of: (1) cancerous (or cancer) cells that proliferate by expressing a mutated tyrosine kinase or overexpression of a receptor tyrosine kinase; (2) benign and malignant cells of other proliferative diseases in which aberrant tyrosine kinase activation occurs; (3) any tumors that proliferate by receptor tyrosine kinases; (4) any tumors that proliferate by aberrant serine/threonine kinase activation; and (5) benign and malignant cells of other proliferative diseases in which aberrant serine/threonine kinase activation occurs.

The phrase “cell growth”, as used herein, for example in the context of “tumor cell growth”, unless otherwise indicated, is used as commonly used in oncology, where the term is principally associated with growth in cell numbers, which occurs by means of cell reproduction (i.e., proliferation) when the rate of the latter is greater than the rate of cell death (e.g., by apoptosis or necrosis), to produce an increase in the size of a population of cells, although a small component of that growth may in certain circumstances be due also to an increase in cell size or cytoplasmic volume of individual cells.

An agent that inhibits cell growth can thus do so by either inhibiting proliferation or stimulating cell death, or both, such that the equilibrium between these two opposing processes is altered and overall results in reduction of the cells load in a subject.

The term “tissue” describes an ensemble of cells, not necessarily identical, but from the same origin, that together carry out a specific function.

The phrase “inhibiting cell growth” describes, as indicated above, altering the equilibrium between cells proliferation and cell death such that a rate of cell death is increased and is higher than the proliferation rate, resulting in a reduced or nullified number of viable cells. Thus, this phrase encompasses reducing or inhibiting proliferation of cells, killing cells, and/or reducing a volume of a tissue formed of the cells (a tumor tissue).

The term “primary tumor” describes a tumor that is at the original site where it first arose.

The term “secondary tumor” describes a tumor that has spread from its original (primary) site of growth to another site, close to or distant from the primary site, and is also referred to herein and in the art as metastasis, or as metastasizing tumor. The term “secondary tumor” as used herein also describes recurrent tumor, which can ne at the original site as the primary tumor and/or at another site, as a metastasizing tumor.

The primary and/or secondary tumor(s) treated according to any of the respective embodiments described herein is preferably a tumor in a respiratory tract of the subject, for example, a lung tumor (e.g., a lung cancer primary tumor) and/or a lung metastasis (secondary tumor). Thus, for example, inhalation of gNO may optionally be regarded as local administration of such a tumor.

When the respiratory tract tumor is a secondary tumor (e.g., lung metastasis), the primary tumor may optionally originate in or on any organ, such as, without limitation, the adrenal gland, bladder, bone, breast, central nervous system (e.g., brain), cervix, gastrointestinal tract (e.g., colon, rectum, small intestine, stomach, esophagus and/or mouth), heart, kidney, liver, lung, ovary, pancreas, parathyroid gland, pituitary gland, prostate, salivary gland, skin, spleen, thymus, thyroid, testicles, urinary tract, uterus, and/or vagina.

The tumor (e.g., respiratory tract tumor) may optionally be malignant or benign. Examples of benign lung tumors treatable according to embodiments of the invention include, without limitation, hemangiomas, acoustic neuromas, neurofibromas, trachomas and pyogenic granulomas.

Herein, the term “malignant” describes a tumor that is not self-limited in its growth, is capable of invading into adjacent tissues, and may be capable of spreading to distant tissues (metastasizing); whereas the term “benign” describes a tumor which is not malignant (i.e. does not grow in an unlimited, aggressive manner, does not invade surrounding tissues, and does not metastasize).

According to some of any of the embodiments described herein, the tumor is a malignant tumor, for example, a malignant cancerous tumor, and the tumor cells are cancer or cancerous cells.

The term “cancer” encompasses malignant and benign tumors as well as disease conditions evolving from primary or secondary tumors, as described herein.

Examples of benign tumors include, without limitation, lipomas, chondromas, adenomas, pilomatricomas, teratomas, and hamartomas.

Cancers treatable according to embodiments of the invention include, but are not limited to, carcinomas, sarcomas, lymphomas, blastomas, and germ cell tumors. Carcinomas include, without limitation, adenocarcinomas (e.g., small cell lung cancer, kidney, uterus, prostate, bladder, ovary and/or colon adenocarcinoma) and epithelial carcinomas.

The primary or secondary tumor (e.g., respiratory tract tumor) may optionally be associated with any solid or non-solid cancer and/or cancer metastasis, including, but is not limiting to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung cancer, nasopharyngeal, breast carcinoma (e.g., ductal breast cancer, invasive intraductal breast cancer, type 4 breast cancer, breast cancer-1, breast cancer-3, breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, cholangiocarcinoma, choriocarcinoma, cystadenocarcinoma), ependymoblastoma, ependymoma, epithelioma, erythroleukemia (e.g., Friend, lymphoblast), fibrosarcoma, fibrous tumor, giant cell tumor, glial tumor, glioblastoma (e.g., multiforme, astrocytoma), glioma, head and neck cancer, hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute-megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, neuroendocrine tumors, oligoastrocytoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, gastric, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, thyoma, and Turcot syndrome with glioblastoma.

In some of any of the respective embodiments described herein, a daily dosage of gNO and/or duration of treatment is selected effective to inhibit growth, kill and/or eradicate cancer cells and/or to stimulate an anti-cancer immune response.

Herein, the phrase “effective to inhibit growth, kill and/or eradicate cancer cells” refers to a reduction of at least 5%, or at least 10%, preferably at least 20%, or at least 30%, or at least 40%, or at least 50%, in volume, weight and/or number of cancer cells in a tumor, and/or to a reduction in cancer cell growth of at least 5%, or at least 10%, preferably at least 20%, or at least 30%, or at least 40%, or at least 50%, as compared to non-treated cancer cells.

The tumor according to any of the respective embodiments described herein may optionally be characterized (e.g., for monitoring efficacy of treatment) by computed tomography, ultrasound, magnetic resonance imaging (MRI), or any other imaging device or imaging involving procedure, such as laparoscopy or bronchoscopy.

In some of any of the respective embodiments described herein, the method and/or treatment further comprises co-administering to the subject an anti-cancer therapy (i.e., other than the gNO administration). In some such embodiments, the gNO administration sensitizes the subject to the anti-cancer therapy, e.g., in a synergistic manner.

Sensitizing a subject to an anti-cancer therapy by co-administering gNO (according to any of the respective embodiments described herein) may optionally allow for a low dosage of anti-cancer therapy (e.g., a lower dosage than an ordinary dosage used in the art) to be efficacious, and/or enhance the efficacy of an ordinary dosage of the anti-cancer therapy. Alternatively or additionally, a subject to an anti-cancer therapy (according to any of the respective embodiments described herein) may optionally allow for an anti-cancer therapy to have greater efficacy against a tumor which is non-responsive to the anti-cancer therapy per se. Enhancement of efficacy may comprise causing an anti-cancer therapy which is ordinarily non-efficacious (e.g., for a given type of tumor) to be efficacious, or causing an anti-cancer therapy with limited efficacy (e.g., for a given type of tumor) to have a greater efficacy (e.g., manifested as a longer life expectancy and/or higher remission rate).

According to an aspect of some embodiments of the invention, there is provided a method of sensitizing cells or tissue of a primary and/or secondary tumor (e.g., a tumor according to any of the respective embodiments described herein) in a subject in need thereof to an anti-cancer therapy, the method comprising co-administering to the subject the anti-cancer therapy and gaseous nitric oxide (gNO), wherein the gNO is administered via inhalation, optionally according to any of the respective embodiments described herein.

In some of any of the embodiments described herein relating to co-administering gNO and an anti-cancer therapy (according to any of the aspects described herein), administration of the gNO may optionally be performed prior to, subsequent to, and/or concomitant with administering the anti-cancer therapy.

In some of any of the embodiments described herein, co-administering gNO allows the administration of the anti-cancer therapy at a sub-therapeutic dosage; which may, for example, reduce the adverse effects of the treatment (e.g., even if the gNO administration itself results in moderate adverse effects).

Herein, the term “sub-therapeutic dosage” or “sub-therapeutic dose” refers to a dosage of an agent which is lower than a dosage of the agent effective (when administered alone) to prevent, alleviate or ameliorate symptoms of a disorder (e.g., a tumor) or prolong the survival of the subject being treated; for example, a dosage lower than a dosage of the agent recognized in the art to be effective (when administered alone) for such a purpose, or a dosage that was shown to be effective (when administered alone) to prevent, alleviate or ameliorate symptoms of a disorder (e.g., a tumor) or prolong the survival of a specific subject.

In other words, the term “sub-therapeutic dosage” or “sub-therapeutic dose” refers to a dosage of an agent which is lower than a therapeutically effective amount of the agent (when administered alone) to the subject, as defined herein.

Determination of a therapeutically effective amount is well within the capability of those skilled in the art. For example, the therapeutically effective amount or dose can be estimated initially from in vitro and cell culture assays. For example, a dose can be formulated in animal models to achieve a desired concentration or titer. Such information can be used to more accurately determine useful doses in humans. The dosage may vary depending upon the dosage form employed and the route of administration utilized. The exact formulation, route of administration and dosage can be chosen by the individual physician in view of the patient's condition.

In exemplary embodiments, a therapeutically effective amount of an anti-cancer therapy is such that results (in combination with gNO administration) in inhibiting the growth of at least 50% of the cancer cells, and/or reduces the volume or weight of the tumor by at least 50%.

Without being bound by any particular theory, it is believed that gNO binds to intracellular thiols which are involved (inter alia) in detoxification (e.g., of electrophiles), such that cancer cells exposed to gNO may be less capable of resisting cytotoxic conditions such as those associated with an anti-cancer therapy.

The gNO may optionally be administered along with any anti-cancer therapy known in the art, for example, comprising an anti-cancer agent (e.g., a chemotherapeutic agent and/or an immune-oncological agent), and/or a radiation treatment.

As used herein, the terms “chemotherapy” or “chemotherapeutic” refer to an agent that reduces, prevents, mitigates, limits, and/or delays the growth of neoplasms or metastases, or kills neoplastic cells directly by necrosis or apoptosis of neoplasms or any other mechanism, or that can be otherwise used, in a pharmaceutically-effective amount, to reduce, prevent, mitigate, limit, and/or delay the growth of neoplasms or metastases in a subject with neoplastic disease (e.g., cancer).

Chemotherapeutic agents include, but are not limited to, fluoropyrimidines, pyrimidine nucleosides, purine nucleosides, anti-folates, platinum agents, anthracyclines/anthracenediones, epipodophyllotoxins, camptothecins (e.g., Karenitecin), hormones, hormonal complexes, antihormonals, enzymes, proteins, peptides and polyclonal and/or monoclonal antibodies, vinca alkaloids, taxanes, epothilones, antimicrotubule agents, alkylating agents, antimetabolites, topoisomerase inhibitors, antivirals, and various other cytotoxic and cytostatic agents.

In some of any of the respective embodiments described herein, the chemotherapeutic agent is an analog of a (pyrimidine or purine) nucleobase, nucleoside and/or nucleotide. Examples of suitable analogs include, without limitation, 5-fluorouracil, floxuridine, 6-azauracil, cytarabine, gemcitabine, thiopurines (e.g., 6-thioguanine, 6-mercaptopurine, or azathioprine), clofarabine, pentostatin, cladribine, and fludarabine. 5-Fluorouracil is an exemplary nucleobase analog.

In some of any of the respective embodiments described herein, the chemotherapeutic agent is an EGFR tyrosine kinase inhibitor. Examples of suitable EGFR tyrosine kinase inhibitors include, without limitation, gefitinib, erlotinib, afatinib, dacomitinib, and osimertinib.

Alternative or additional chemotherapeutic agents that may optionally be administered according to any of the respective embodiments include, but are not limited to acivicin, aclarubicin, acodazole, acronine, adozelesin, aldesleukin, altretamine, ambomycin, ametantrone, aminoglutethimide, amsacrine, anastrozole, anthramycin, asparaginase, asperlin, azacitidine, azetepa, azotomycin, batimastat, benzodepa, bicalutamide, bisantrene, bisnafide, bizelesin, bleomycin, brequinar, bropirimine, busulfan, cactinomycin, calusterone, caracemide, carbetimer, carboplatin, carmustine, carubicin, carzelesin, cedefingol, chlorambucil, cirolemycin, cisplatin, cladribine, crisnatol, cyclophosphamide, cytarabine, dacarbazine, dactinomycin, daunorubicin, decitabine, dexormaplatin, dezaguanine, diaziquone, docetaxel, doxorubicin, droloxifene, dromostanolone, duazomycin, edatrexate, eflornithine, elsamitrucin, enloplatin, enpromate, epipropidine, epirubicin, erbulozole, esorubicin, estramustine, etanidazole, etoposide, etoprine, fadrozole, fazarabine, fenretinide, floxuridine, fludarabine, fluorouracil, flurocitabine, fosquidone, fostriecin, gemcitabine, hydroxyurea, idarubicin, ifosfamide, ilmofosine, interferon alfa-2a, interferon alfa-2b, interferon alfa-n1, interferon alfa-n3, interferon beta-Ia, interferon gamma-Ib, iproplatin, irinotecan, lanreotide, letrozole, leuprolide, liarozole, lometrexol, lomustine, losoxantrone, masoprocol, maytansine, mechlorethamine, megestrol, melengestrol, melphalan, menogaril, mercaptopurine, methotrexate, metoprine, meturedepa, mitindomide, mitocarcin, mitocromin, mitogillin, mitomalcin, mitomycin, mitosper, mitotane, mitoxantrone, mycophenolic acid, nocodazole, nogalamycin, ormaplatin, oxisuran, paclitaxel, pegaspargase, peliomycin, pentamustine, peplomycin, perfosfamide, pipobroman, piposulfan, piroxantrone, plicamycin, plomestane, porfimer, porfiromycin, prednimustine, procarbazine, puromycin, pyrazofurin, riboprine, rogletimide, safingol, semustine, simtrazene, sparfosate, sparsomycin, spirogermanium, spiromustine, spiroplatin, streptonigrin, streptozocin, sulofenur, talisomycin, tecogalan, tegafur, teloxantrone, temoporfin, teniposide, teroxirone, testolactone, thiamiprine, thioguanine, thiotepa, tiazofurin, tirapazamine, topotecan, toremifene, trestolone, triciribine, trimetrexate, triptorelin, tubulozole, uracil mustard, uredepa, vapreotide, verteporfin, vinblastine, vincristine, vindesine, vinepidine, vinglycinate, vinleurosine, vinorelbine, vinrosidine, vinzolidine, vorozole, zeniplatin, zinostatin, zorubicin, and any pharmaceutically acceptable salts thereof.

As used herein, the term “immune-oncological agent” refers to an agent that induces an immune response that reduces, prevents, mitigates, limits, and/or delays the growth of neoplasms or metastases in a subject with neoplastic disease (e.g., cancer), but which does not necessarily have anti-neoplastic or anti-metastatic effect in the absence of an immune response (e.g., in vitro).

Examples of immune-oncological agents include, without limitation, immune checkpoint inhibitors (e.g., a PD1 inhibitor and/or a PDL1 inhibitor), chimeric antigen receptor T cells (CAR-T cells), and adjuvants (e.g., CpG DNA, interferon and/or saponin).

In some of any of the respective embodiments described herein, the immune-oncological agent is a PD1 inhibitor and/or a PDL1 inhibitor, for example, an antibody (e.g., monoclonal antibody) targeting PD1 or PDL1.

Examples of suitable PD1 inhibitors include, without limitation, pembrolizumab, nivolumab, cemiplimab, spartalizumab, sintilimab, tislelizumab, toripalimab, dostarlimab, JTX-4014, INCMGA00012, AMP-224, and AMP-514.

Examples of suitable PDL1 inhibitors, include, without limitation, atezolizumab, durvalumab, avelumab, KN035, CK-301, AUNP12, CA-170 and BMS-986189.

Additional anti-cancer agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division).

Without being bound by any particular theory, it is believed that administration of gNO by inhalation can induce heat shock protein expression within and/or near a tumor, and/or release of antigens from damaged or destroyed tumor cells; either of which may enhance an immune response towards the tumor (e.g., via activation of anti-tumor T cells). Such an immune response may be, for example, an immune response associated with an activity of an immune-oncological agent described herein (according to any of the respective embodiments) or may be a natural immune response in a subject which ordinarily complements any anti-cancer therapy (e.g., a chemotherapeutic agent and/or radiation treatment). It is further believed that enhancement of such an immune response may induce regression of similar tumors not exposed to the gNO, for example, wherein an induced immune response towards a secondary lung tumor in a lung facilitates regression of a primary and/or secondary tumor outside the lung.

In some of any of the respective embodiments described herein, the chemotherapeutic agent and/or immune-oncological agent is administered by injection, e.g., intravenous injection.

In some of any of the respective embodiments described herein, the chemotherapeutic agent and/or immune-oncological agent is administered via inhalation, e.g., wherein the agent(s) is mixed with the gNO and/or wherein the agent and gNO are administered via inhalation separately.

In order to enhance treatment of the cancer, the present invention further envisions administering to the subject an additional therapy such as radiotherapy, chemotherapy, phototherapy and photodynamic therapy, surgery, nutritional therapy, ablative therapy, combined radiotherapy and chemotherapy, brachiotherapy, proton beam therapy, immunotherapy, cellular therapy and photon beam radiosurgical therapy. Analgesic agents and other treatment regimens are also contemplated.

According to an aspect of some embodiments of the invention, there is provided a chemotherapeutic agent and/or immune-oncological agent (e.g., according to any of the respective embodiments described herein) for use in the treatment of a primary and/or secondary tumor (e.g., the treatment comprising inhibiting growth of cells or tissue of the tumor in a subject in need thereof), wherein the treatment further comprises administering gNO via inhalation according to any of the respective embodiments described herein.

According to an aspect of some embodiments of the invention, there is a use of a chemotherapeutic agent and/or immune-oncological agent (e.g., according to any of the respective embodiments described herein) in the manufacture of a medicament for the treatment of a primary and/or secondary tumor (e.g., the treatment comprising inhibiting growth of cells or tissue of the tumor in a subject in need thereof), wherein the treatment further comprises administering gNO via inhalation according to any of the respective embodiments described herein.

It is expected that during the life of a patent maturing from this application many relevant active agents and anti-cancer therapies will be developed, and the scope of the terms “anti-cancer therapy”, “chemotherapeutic agent”, “immune-oncological agent”, “radiation treatment” and “agent suitable for treating methemoglobinemia” are intended to include all such new technologies a priori.

According to some embodiments of the present invention, in any of the methods of treatment presented herein, the gNO administration can be effected by an inhalation device which includes, without limitation, a stationary inhalation device, a portable inhaler, a metered-dose inhaler and an intubated inhaler.

An inhaler, according to some embodiments of the present invention, can generate spirometry data and adjust the treatment accordingly over time as provided, for example, in U.S. Pat. No. 5,724,986 and WO 2005/046426. The inhaler can modulate the subject's inhalation waveform to target specific lung sites. According to some embodiments of the present invention, a portable inhaler can deliver both rescue and maintenance doses of gNO at subject's selection or automatically according to a specified regimen.

According to some embodiments of the present invention, an exemplary inhalation device may include a delivery interface adaptable for inhalation by a human subject.

According to some embodiments of the present invention, the delivery interface includes a mask, mouthpiece or other system (e.g., nasal cannula) for delivery of the gNO to a respiratory organ of the subject.

Alternatively or additionally, at least a portion of the subject is present in an atmospherically enclosure containing the gNO. An atmospherically controlled enclosure includes, without limitation, a head enclosure (bubble), a full body enclosure (e.g., a full body hood) or a room, wherein the atmosphere filling the enclosure can be controlled by flow, by a continuous or intermittent content exchange or any other form of controlling the gaseous mixture content thereof.

According to some embodiments of the present invention, the inhalation device further includes a gas analyzer positioned in proximity to the delivery interface for measuring the concentration of gNO, oxygen and/or NOx species (e.g., NO₂) flowing to the delivery interface, wherein the analyzer is optionally in communication with a controller.

The gNO may be provided by an external source, for example a reservoir (e.g., gas cylinder) of gNO (optionally mixed with a carrier gas) or a chemical generator of gNO, for example, any NO-oxide producing compound, composition or substance (e.g., wherein the compound, composition or substance generates NO upon a thermal, chemical, ultrasonic, and/or electrochemical reaction). An example of a suitable NO generator is described in U.S. Pat. No. 9,573,110, which is incorporated herein by reference.

The gNO may optionally be passed through a filter (e.g., a soda lime filter) configured for removing at least a portion of NOx species (e.g., NO₂) from the gNO.

Dosage and/or flow rate may optionally be controlled using a pressure regulator, flow meter and/or one-way valve to control the amount of gNO flowing from the gNO source (e.g., wherein disconnecting the regulator or flow meter from the gNO source locks the valve, thereby preventing gas release from the source) and/or by controlled evacuation and/or purging (e.g., with an inert gas such as nitrogen) of gNO (e.g., in a pulsed or continuous manner) from any portion of the inhalation device (e.g., pressure regulator, flow meter and/or delivery lines), optionally wherein the evacuated/purged gNO is directed to an evacuation cylinder. A detector may optionally be used to monitor levels of NO and/or NOx species (e.g., NO₂).

According to some embodiments of the present invention, subjecting the subject to the method described herein is carried out by use of an inhalation device which can be any device which can deliver the mixture of gases containing gNO to a respiratory organ of the subject. An inhalation device, according to some embodiments of the present invention, includes, without limitation, a stationary inhalation device comprising tanks or cylinders, gauges, tubing, a mask, controllers, valves and the like; a portable inhaler (inclusive of the aforementioned components), a metered-dose inhaler, a an atmospherically controlled enclosure, a respiration machine/system and an intubated inhalation/respiration machine/system.

The following describes exemplary configurations of an inhalation device and of methods employing same in the context of the methods and uses as described herein in any of the respective embodiments.

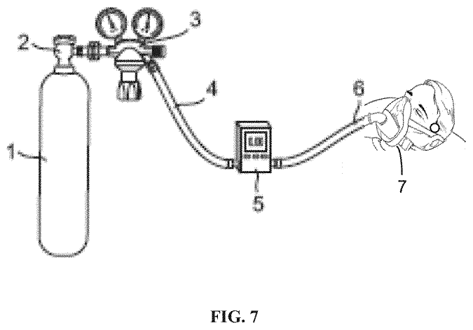

Referring in this regard to the figures, FIG. 7 is a schematic illustration of a delivery system in which gaseous NO is stored in a cylinder 1 with a valve 2 such as, but not limited to, a one-way valve, on top of it, which is connected to a pressure regulator 3.

Cylinder 1 is optionally and preferably disposable. This is particularly advantageous when the gas is toxic, as in the case of gNO, so that the disposable cylinder can be connected to the delivery system immediately before treatment, and disposed immediately after treatment, thus reducing the time at which the toxic substance is in the treating or operating room.

A digital flow controller 5 is connected to the pressure regulator 3 by a designated gas tubing 4. gNO is delivered to delivery interface 7 which is configured for delivering a gas to a subject's face—for example, a face mask and/or a nasal and/or oral cannula—by a gas tubing 6 that is connected to delivery interface 7 at its distal end and to flow controller 5 at its proximal end. Any of the pressure regulator 3, the digital flow controller 5, and/or the tubing lines 4 and/or 6 may optionally comprise, or be operatively connected to, an NO₂ filter (not shown) configured to lower NO₂ levels upon passage of gas through the filter. The delivery system may optionally further comprise a gas analyzer (not shown) configured for monitoring (e.g., continuously monitoring) NO, NO₂ and/or O₂ levels in tubing lines 4 and/or 6; for example, being connected to tubing lines 4 and/or 6 (e.g., via a sampling line) and/or being comprised by digital flow controller 5.

gNO is delivered to delivery interface 7 while excess gas is optionally evacuated by suctioning through a designated gas tubing to an evacuation system that is preferably connected to the medical center pipe to allow releasing of the gas outside (not shown). The suctioning of the gas can be done in a pulsed or continuous manner, preferably synchronized with gNO administration. Purging of the gNO delivery system; including the pressure regulator 3, the digital flow controller 5, delivery interface 7 and the tubing lines 4 and 6 can be performed before and/or after treatment. Nitrogen can be used as an exemplary purge gas for purging the system. The purge gas can be introduced from a separate cylinder (not shown).

It is expected that during the life of a patent maturing from this application many relevant techniques and apparatuses suitable for administering a gas such as NO will be developed, and the scope of all references herein to administration of gaseous NO is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “treating” refers to inhibiting or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

In Vitro NO Treatment:

10,000-20,000 cells from a cancer cell line were plated onto a 96-well plate. After an overnight incubation at 37° C. and 5% CO₂, the cell culture medium was removed, and the plate was placed inside an exposure chamber made of acrylic glass, which was closed and sealed with paraffin wax tape. A gas delivery line was inserted from the top of the chamber. NO supplied from 800 ppm NO cylinders, as well as air and oxygen, were delivered to the chamber at a gas flow rate of 1 liter per minute. NO was diluted to the indicated concentration and the final oxygen concentration inside the chamber was adjusted to about 21%, to avoid cell death due to lack of oxygen. Gas exposure lasted for the indicated time period, for one to seven exposure cycles. Gas exchange during this time was permitted via an outlet hole located close to the bottom of the chamber. Cancer cells exposed to air served as a control group. Cell viability was determined using an XTT (2,3-bis-(2-methoxy-4-nitro-5-sulfophenyl)-2H-tetrazolium-5-carboxanilide) assay, whereby conversion of the XTT by viable cells to the corresponding formazan dye was quantified by spectrophotometry.

In Vivo Cancer Model and NO Treatment:

CT26 cells were harvested using trypsin (Biological Industries, Israel). The cells were then centrifuged at 1,200 rotations per minute for 7 minutes. The numbers of viable and dead cells were recorded. The pellet was re-suspended in HBSS (Hank's Balanced Salt Solution) at a concentration of 10×10⁶ cells (both viable and dead) per 1 ml HBSS. The suspension of CT26 cells in HBSS was inoculated to the tail vein of Balb/c mice (intravenously) at a volume of 200 μl per mouse (total of 2,000,000 cells per mouse). About five minutes before cell injection, mice were exposed to red light to enable expansion of the vein. After injection, the mice were placed back in their cages.

To administer gaseous NO by inhalation, the mouse cage was sealed with a plastic coverage, and gas tubing was placed inside the cage through one of the eight pores placed near by the bottom of the cage. 200 (±10) ppm NO mixed with air, including a final concentration of 21% (±1%) oxygen, was delivered to the cage for 1.5 hours, twice a day, with 4 hour of rest between inhalation. As a control, air was administered without NO. NO, NO₂ and oxygen levels were monitored every 15-30 minutes during inhalation sessions.

Mouse clinical status was assessed using a score table that included a specific assay for lung metastatic burden. When a mouse reached a score of 12, it was sacrificed, and the death date was recorded.

### Example 1

### Safety Study for Gaseous NO

The effect of intermittent inhalation of NO at a concentration of 250-400 ppm was tested for 30 days in healthy dogs and for 12 days in healthy rats. No macroscopic or microscopic effect of the NO inhalation was observed, indicating that repeated inhalation of NO at a concentration on the order of hundreds of ppm is nontoxic.

### Example 2

### Effect of Gaseous NO on Cancer Cells In Vitro

CT26 colon cancer cells (a mouse colon cancer line) were grown in 100 μl RPMI (Roswell Park Memorial Institute medium)-based tissue culture medium, supplemented with 1% streptomycin-penicillin and 10% fetal bovine serum, and plated 1 day before exposure to NO in 10 ml RPMI-based tissue culture medium; and then exposed to 400 ppm NO for 1 hour, or to 200 ppm NO for one or more cycles of 15 minutes (separated by 30 minutes of rest at 37° C.), using the procedures described hereinabove. Immediately after each exposure, 100 μl of the abovementioned supplemented RPMI-based tissue culture medium was added to each well and plated were stored at 37° C. and 5% CO₂.

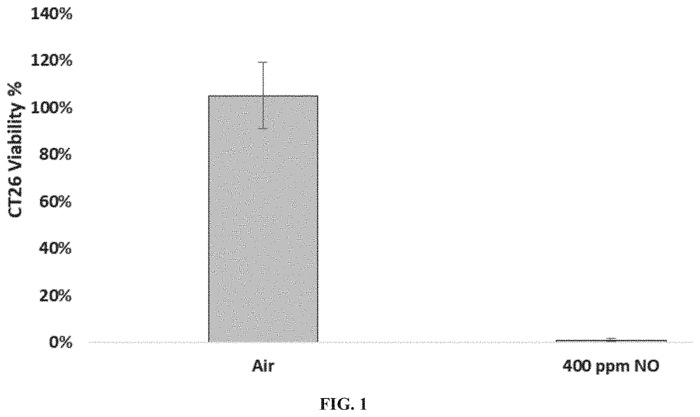

As shown in FIG. 1, CT26 cells were not viable after exposure to 400 ppm NO for 1 hour, whereas air did not harm the cells.

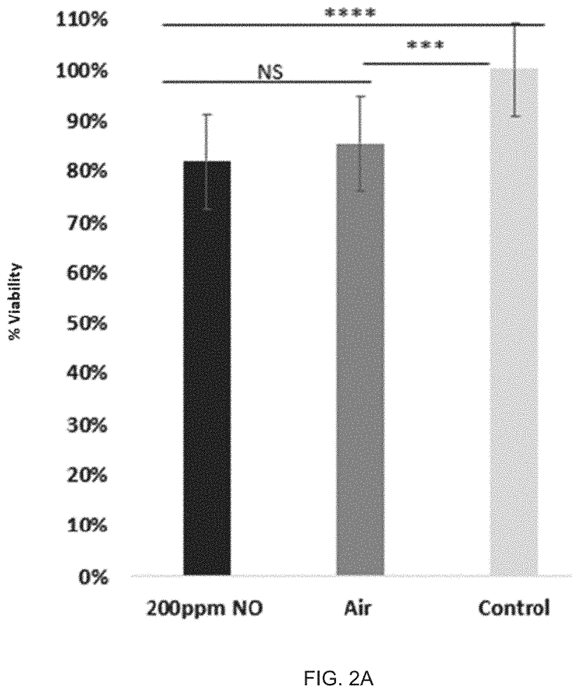

As shown in FIGS. 2A-2C, a single 15 minute exposure to 200 ppm NO had little effect on CT26 cell viability (FIG. 2A), whereas CT26 cell viability was decreased to about 40% after 4 cycles of exposure for 15 minutes to 200 ppm NO (FIG. 2B). However, exposure to seven cycles of exposure for 15 minutes to 200 ppm NO (FIG. 2C) did not produce an additional reduction in viability, as compared to exposure to four cycles.

Furthermore, LLC1 lung cancer cells (a mouse lung cancer line) were exposed to 200 ppm NO for 15 or 30 minutes, using the procedures described hereinabove.

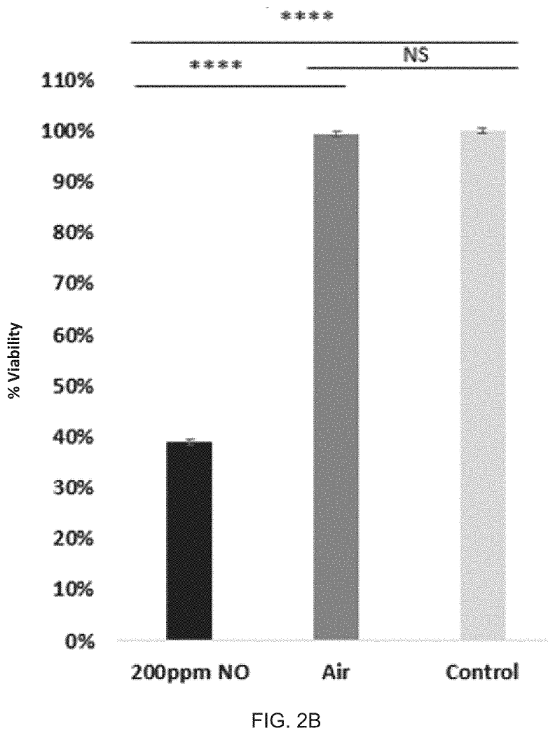

As shown in FIG. 3, 41% of LLC1 cells were not viable after exposure to 200 ppm NO for 15 minutes, and 65% of LLC1 cells were not viable after exposure to 200 ppm NO for 30 minutes.

These results indicate that gNO can kill cancer cells at concentrations suitable for inhalation.

### Example 3

### Effect of Gaseous NO in Combination with Chemotherapeutic Agent on Cancer Cells In Vitro

CT26 colon cancer cells were exposed to 1 μM of the chemotherapeutic agent 5-fluorouracil (5FU), in addition to 200 ppm NO for 30 minutes, using the procedures described hereinabove, except that after overnight incubation at 37° C. and 5% CO₂, the 5FU was added to the cell culture medium, and the cells were subjected to a second overnight incubation prior to being placed within the exposure chamber, as described therein.

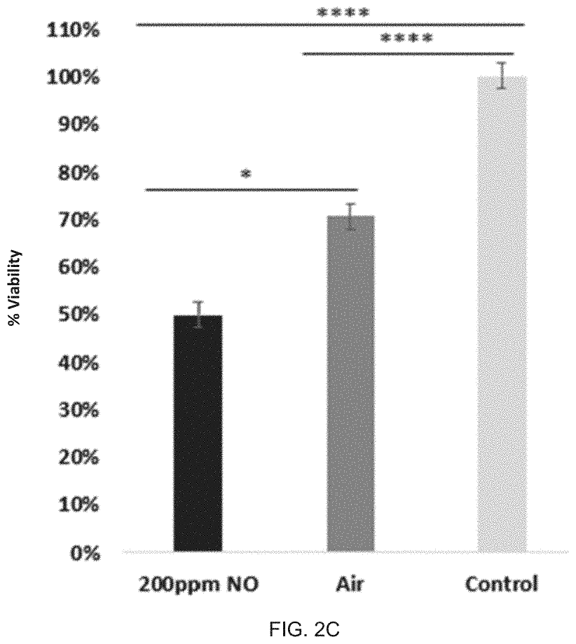

As shown in FIG. 4, 5FU alone had little effect on the CT26 cells, whereas exposure to 200 ppm NO for 30 minutes reduced CT26 cell viability by 51%, and the combination of 5FU and exposure to 200 ppm NO for 30 minutes reduced CT26 cell viability by 71%.

In another experiment, CT26 cells were exposed to 0.5-50 μM 5FU and 4 cycles of 200 ppm NO for 10 minutes (separated by 30 minutes of rest at 37° C.), using the procedures described hereinabove, except that the 5FU was added immediately after the last exposure to NO (or air), followed by incubation for about 24 hours.

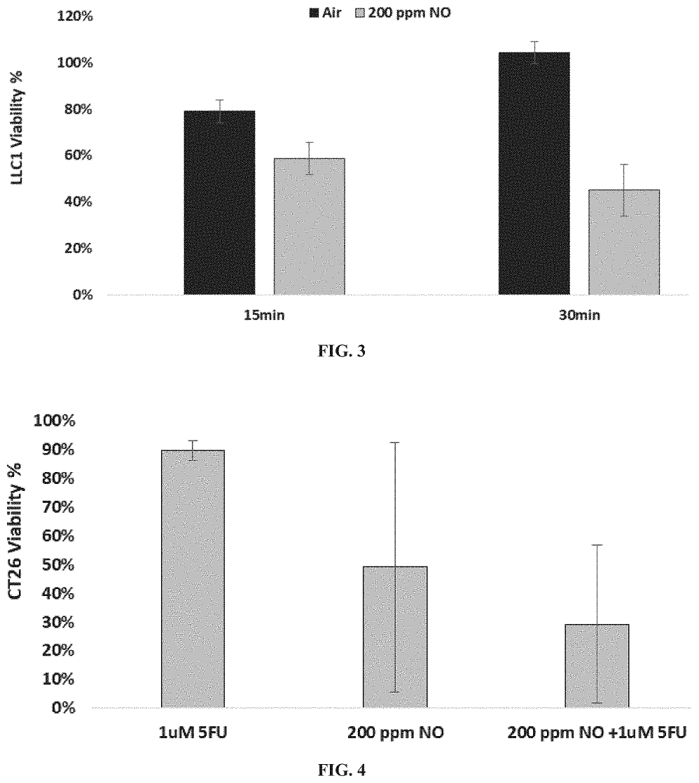

As shown in FIG. 5, all tested concentrations of 5FU reduced CT26 cell viability to less than 40% when combined with NO treatment.

These results indicate that gNO at concentrations suitable for inhalation can enhance the ability of chemotherapy to kill cancer cells.

### Example 4

### Effect of Gaseous NO with Chemotherapeutic Agent on Cancer in Animal Model

In order to investigate the effect of NO inhalation on cancer in vivo, a colon cancer lung metastasis mouse model was used.

Mice were injected intravenously with CT26 colon cancer cells on day 0, and on each of days 3-18, the mice were administered via inhalation 200 ppm NO (or air, as a control), according to procedures described in the Materials and Methods section hereinabove. On days 4, 11 and 16, some of the mice received an intra-peritoneal injection of a low dose (20 mg/kg) of 5-fluorouracil (5FU), which was freshly prepared prior to each inoculation in sterile saline solution.

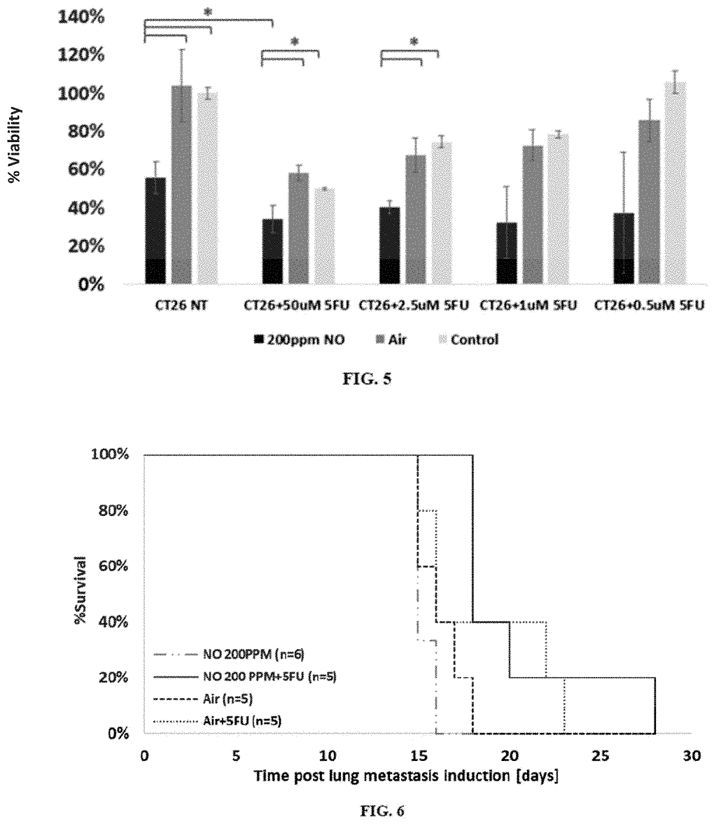

As shown in FIG. 6, treatment with both gaseous NO and 5FU resulted in the highest degree of survival in mice.

These results further indicate that gNO at concentrations suitable for inhalation can enhance the ability of chemotherapy to kill cancer cells.

### Example 5

### Additional Studies of Effect of Gaseous NO in Combination with Chemotherapeutic Agent on Cancer Cells In Vitro

Cancer cells are exposed to 1-1,000 ppm NO for 1-360 minutes for 1-5 cycles per day for 1-10 days. Various chemotherapeutic agents (e.g., other than 5-fluorouracil) at various doses are added and cell viability is monitored (e.g., according to procedures such as described hereinabove). Nitrogen gas and/or absence of chemotherapeutic agent are optionally used as a control.

### Example 6

### Additional Studies of Effect of Gaseous NO in Combination with Chemotherapeutic Agent on Cancer In Vivo

Cancer cells are injected to the tail vein of mice in order to induce lung tumors and/or metastases (e.g., according to procedures described hereinabove). The mice are then administered 1-1,000 ppm NO via inhalation for 1-360 minutes for 1-5 cycles per day for 1-100 days, optionally according to procedures described hereinabove. One or more chemotherapeutic agent is injected 1-times during the course of the NO treatment. Naive mice and/or mice administered nitrogen gas and/or not administered chemotherapeutic agent are optionally used as a control.

Lung tumor and/or metastasis burden is then assessed, and optionally also the anti-tumor immune response via a challenge assay (whereby cancer cells are injected into a treated mouse to determine whether a tumor develops) and monitoring immune cell and/or antibody levels.

### Example 7

### Effect of Gaseous NO on PDL1 in Cancer Cells

The immune checkpoint ligand PDL1 (Programmed death-ligand 1) can block the immune system from attacking cancer cells, and is therefore a target of PDL1 inhibitor anticancer drugs.

The potential effects of NO on PDL1 inhibitor therapy is investigated by exposing PDL1-negative cells or wild type (PDL1-positive) cancer cells to 1-1,000 ppm NO for 1-360 minutes for 1-5 cycles per day for 1-10 days. The level of PDL1 expression is evaluated using fluorescently labeled anti-PDL1 antibodies, monitored via a flow cytometer. In addition, cell viability is monitored (e.g., according to procedures such as described hereinabove). Exposure to nitrogen gas and/or absence of treatment are optionally used as a control.

### Example 8

### Effect of Gaseous NO in Combination with Anti-PDL1 Antibodies on Cancer In Vivo

PDL1-negative cells or wild type (PDL1-positive) cancer cells are injected to the tail vein of mice in order to induce lung tumors and/or metastases (e.g., according to procedures described hereinabove). The mice are then administered 1-1,000 ppm NO via inhalation for 1-360 minutes for 1-5 cycles per day for 1-100 days, optionally according to procedures described hereinabove. Anti-PDL1 antibody is injected 1-20 times during the course of the NO treatment. Naive mice and/or mice exposed to nitrogen gas instead of NO (or otherwise not exposed to NO) and/or not administered anti-PDL1 antibody are optionally used as a control.

Lung tumor and/or metastasis burden is then assessed, and optionally also the anti-tumor immune response via a challenge assay (whereby cancer cells are injected into a treated mouse to determine whether a tumor develops) and monitoring immune cell and/or antibody levels.

### Example 9

### Effect of Gaseous NO on EGFR in Cancer Cells

EGFR (epidermal growth factor receptor) is a tyrosine kinase implicated in many cancer types, and is therefore a target of EGFR tyrosine kinase inhibitor (TKI) anticancer drugs.

The potential effects of NO on EGFR-TKI therapy is investigated by exposing EGFR-negative cells or wild type (EGFR-positive) cancer cells to 1-1,000 ppm NO for 1-360 minutes for 1-5 cycles per day for 1-10 days. The level of EGFR expression is evaluated using fluorescently labeled anti-EGFR antibodies, monitored via a flow cytometer. In addition, cell viability is monitored (e.g., according to procedures such as described hereinabove). Exposure to nitrogen gas and/or absence of treatment are optionally used as a control.

### Example 10

### Effect of Gaseous NO in Combination with EGFR Tyrosine Kinase Inhibitor on Cancer In Vivo

EGFR-negative cells or wild type (EGFR-positive) cancer cells are injected to the tail vein of mice in order to induce lung tumors and/or metastases (e.g., according to procedures described hereinabove). The mice are then administered 1-1,000 ppm NO via inhalation for 1-360 minutes for 1-5 cycles per day for 1-100 days, optionally according to procedures described hereinabove. An EGFR tyrosine kinase inhibitor is injected 1-20 times during the course of the NO treatment. Naive mice and/or mice exposed to nitrogen gas instead of NO (or otherwise not exposed to NO) and/or not administered EGFR tyrosine kinase inhibitor are optionally used as a control.

Lung tumor and/or metastasis burden is then assessed, and optionally also the anti-tumor immune response via a challenge assay (whereby cancer cells are injected into a treated mouse to determine whether a tumor develops) and monitoring immune cell and/or antibody levels.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

While this invention has been particularly shown and described with references to preferred embodiments thereof, it will be understood by those skilled in the art that various changes in form and details may be made therein without departing from the scope of the invention encompassed by the appended claims.

1. A method of inhibiting growth of cells or tissue of a primary and/or secondary tumor in a respiratory tract of a subject in need thereof, the method comprising administering to the subject gaseous nitric oxide (gNO) via inhalation for at least 1 second per day, wherein a concentration of gNO is in a range of from about 1 ppm to about 1,000 ppm.

2. The method of claim 1, wherein said concentration of gNO is in a range of from about 10 ppm to about 600 ppm.

3. The method of claim 1, comprising administering gNO for at least 10 minutes per day.

4. The method of claim 1, comprising administering gNO for no more than about 600 minutes per day.

5. The method of claim 1, wherein said gNO is mixed with a carrier gas, and a volumetric flow of said gNO mixed with said carrier gas is from about 1 liter per minute to about 100 liters per minute.

6. The method of claim 1, being effected for a time period in a range of from 1 to 100 days.

7. The method of claim 1, comprising administering gNO from 1 to 4 times per day.

8. The method of claim 1, wherein a product of said concentration of gNO and a daily time of inhalation of gNO is in a range of from about 0.0027 ppm·hour to about 6000 ppm·hour.

9. The method of claim 1, further comprising co-administering to the subject an anti-cancer therapy.

10. The method of claim 9, wherein said anti-cancer therapy is selected from the group consisting of a chemotherapeutic agent, an immune-oncological agent, and a radiation treatment.

11. The method of claim 9, wherein said anti-cancer therapy is administered at a sub-therapeutic dosage.

12. The method of claim 1, wherein said primary and/or secondary tumor in a respiratory tract is a lung cancer tumor and/or a lung metastasis.

13. The method of claim 1, further comprising administering an agent suitable for treating methemoglobinemia.

14-25. (canceled)

26. A method of sensitizing cells or tissue of a primary and/or secondary tumor in a respiratory tract of a subject in need thereof to an anti-cancer therapy, the method comprising co-administering to the subject the anti-cancer therapy and gaseous nitric oxide (gNO), said gNO being administered via inhalation for at least 1 second per day, wherein a concentration of gNO is in a range of from about 1 ppm to about 1,000 ppm.

27. The method of claim 26, wherein said concentration of gNO is in a range of from about 10 ppm to about 600 ppm.

28. The method of claim 26, comprising administering gNO for at least 10 minutes per day.

29. The method of claim 26, comprising administering gNO for no more than about 600 minutes per day.

30. The method of claim 26, wherein said gNO mixed is mixed with a carrier gas, and a volumetric flow of said gNO mixed with said carrier gas is from about 1 liter per minute to about 100 liters per minute.

31. The method of claim 26, comprising administering gNO for a time period in a range of from 1 to 100 days.

32. The method of claim 26, comprising administering gNO from 1 to 4 times per day.

33. The method of claim 26, wherein a product of said concentration of gNO and a daily time of inhalation of gNO is in a range of from about 0.0027 ppm·hour to about 6000 ppm·hour.

34. The method of claim 26, wherein said anti-cancer therapy is selected from the group consisting of a chemotherapeutic agent, an immune-oncological agent, and a radiation treatment.

35. The method of claim 26, wherein said primary and/or secondary tumor in a respiratory tract is a lung cancer tumor and/or a lung metastasis.

36. The method of claim 26, further comprising administering an agent suitable for treating methemoglobinemia.

37. (canceled)
